(19) United States
(12) Patent Application Publication
(10) Pub. No.: US 2010/0209539 A1
(43) Pub. Date: Aug. 19, 2010

(54) COMPOSITIONS AND METHODS OF USING SAME FOR TREATMENT OF A DISEASE OR DISORDER OF THE EYE AND/OR THE ADNEXA OF THE EYE

(75) Inventor: Jan Renneberg, Virum (DK)

Correspondence Address: WEINGARTEN, SCHURGIN, GAGNEBIN & LEBOVICI LLP, TEN POST OFFICE SQUARE, BOSTON, MA, 02109, US

(73) Assignee: OCUMEDIC APS, Copenhagen K (DK)

(21) Appl. No.: 12/515,996

(22) PCT Filed: Nov. 22, 2007
(86) PCT No.: PCT/DK07/50173

§ 371 Date: Aug. 6, 2009

(30) Foreign Application Priority Data
Nov. 22, 2006 (DK) PA 2006 01534

(51) Int. Cl. A61K 33/22; A61K 31/7048; A61P 27/02; A61P 31/12; A61P 31/04; A61P 27/14

(52) U.S. Cl. 424/660; 514/27

(57) ABSTRACT

The present invention relates to compositions comprising at least one flavonoid for the treatment or amelioration of a disease or disorder of the eye and/or the adnexa of the eye in an animal subject, including a human being. More particularly, this invention relates to a composition for the treatment of conjunctivitis, keratoconjunctivitis sicca, and blepharitis. The invention furthermore relates to a pharmaceutical composition comprising at least one flavonoid, such as for example a topical formulation. The source of the flavonoids may be, but are not restricted to, flavonoids extracted from citrus plants. The compositions may furthermore optionally be used in combination with an eyecleaner or eyewash, which may comprise at least one flavonoid.

### FIELD OF INVENTION

The present invention relates to treatment of a disease or disorder of the eye and adnexa of the eye in an animal subject, including a human being. More particularly, this invention relates to a composition for the treatment of conjunctivitis, keratoconjunctivitis sicca, and blepharitis. The invention furthermore relates to a pharmaceutical composition.

### BACKGROUND OF INVENTION

Conjunctiva refers to the moist membrane that lines the eyelids and covers the exposed surface of the sclera. The conjunctiva is kept moist by the lacrimal gland. In addition, the lacrimal gland floods the eye with tears when the sensitive conjunctiva becomes irritated. Conjunctivitis, one of the world's most common eye diseases, refers to a broad group of conditions presenting as inflammation of the conjunctiva. Conjunctivitis can be hyperacute, acute or chronic in presentation and classified as infectious or non-infectious. Conjunctivitis is the most common cause of the conditions with the clinical term “red eye”.

Non-infectious conjunctivitis is associated with dry eyes, allergic conditions induced by pollen or grass (allergic conjunctivitis), exposure to an wide variety of chemical substances or gasses (chemical conjunctivitis), e.g chlorine or hydrochloric acid fumes, splash injury of household or industrial chemicals, toxins (toxic conjunctivitis, thermal and ultraviolet burns), or caused by an underlying disease (e.g Sjoegren's syndrome, Crohn's disease, ulcerative colitis or rheumatoid arthritis). Contact lens wearers may develop conjunctivitis as a direct consequence of the lens itself or due to allergens trap on the lenses.

Infectious conjunctivitis account for the majority conjunctivitis cases and may be classified as viral and bacterial conjunctivitis. Adenovirus is by far the most common cause of viral conjunctivitis, although the condition can also be caused by other viruses including herpes simplex virus. Bacterial conjunctivitis accounts for the majority of all cases of infectious conjunctivitis and is highly contagious and usually caused by staphylococci, pneumococci, streptococci or chlamydia trachomatis.

The typical symptoms common to all forms of conjunctivitis are redness of the eye, irritation and excess tearing. The appropriate treatment of conjunctivitis depends on the cause of the condition. For viral conjunctivitis there is no cure. However supportive treatment includes cool compresses and artificial tears. In severe cases, topical steroid drops may be prescribed to reduce the discomfort from inflammation. Allergic conjunctivitis is also treated with cool compresses and artificial tears. In severe cases of allergic conjunctivitis non-steroidal anti-inflammatory medications, antihistamines, and may be prescribed. Persistent allergic conjunctivitis may require treatment with topical steroid drops.

Keratoconjunctivitis refers to an inflammation of the cornea and conjunctiva. Keratoconjunctivitis sicca, also known as keratitis sicca, xerophthalmia, dry eye syndrome (or simply dry eyes) is the worlds most common eye disease and refers to a condition where the inflammation due to dryness of the eye. The typical symptoms of keratoconjunctivitis sicca are discomfort in the eye, burning and a sandy-gritty eye irritation, sensation of a foreign body in the eye. This soreness can range from mild to severe. In severe cases keratoconjunctivitis sicca may ultimately lead to ocular surface disease causing permanent structural damage to the cornea. The disease is caused by decreased tear production or increased tear film evaporation and it is more common with older age, due to age related reduction in tear production resulting in hypertonic tears. Although less frequently, keratoconjunctivitis sicca can be associated with systemic diseases such as Sjögren's syndrome, systemic lupus erythematosus, rheumatoid arthritis, scleroderma, sarcoidosis, amyloidosis, hypothyroidism, and deficiency of vitamin A. Keratoconjunctivitis sicca is also a common disease in animals including cats and dogs. In dogs most cases are caused by a genetic predisposition, but chronic conjunctivitis, canine distemper, and drugs including sulfasalazine and trimethoprim-sulfonamide may also cause the disease. The best available treatments for keratoconjunctivitis sicca are designed to re-hydrate the tears and eye surface, and include hypotonic, electrolyte-balanced tears (artificial tears), oral nutritional supplements of omega-3, punctal plugs, and moist chamber spectacles. The inflammation that occurs in response to tears film hypertonicity can be suppressed by mild topical steroids or in the recent years with immunosuppressants such as cyclosporin.

Sjoegren's syndrome is a chronic systemic inflammatory disorder characterized by lymphocytic infiltration of exocrine glands, especially the lacrimal and salivary glands. Sjoegren's syndrome is associated with conjunctivitis sicca. The average age of onset is late 40s although Sjoegren's occurs in all age groups in both women and men. Nine out of ten Sjoegren's patients are women. It is estimated to strike as many as 4 million people in the United States alone making it the second most common autoimmune rheumatic disease. There is no known cure for Sjoegren's syndrome and no specific treatment to restore gland secretion. Treatment of Sjoegren's syndrome associated keratoconjunctivitis sicca is symptomatic and supportive and includes the moisture replacement therapies, punctual plugs, and moist chamber spectacles used for keratoconjunctivitis sicca.

Rosacea is a chronic inflammatory skin disease and ocular roseacea is a manifestation of rosacea affecting the eyes and eyelids. The symptoms of ocular roseacea are foreign body sensation, burning or stinging, dryness, itching, sensitivity to light, and blurred vision. There is no cure for ocular roseacea. Supportive treatment includes cool compresses and artificial tears

Another very common eye disease is blepharitis, which is a condition causing inflammation of the eyelid and eyelashes. Blepharitis is classified as anterior blepharitis posterior blepharitis. Anterior blepharitis affects the outside front of the eyelid, where the eyelashes are attached. The two most common causes of anterior blepharitis are bacteria (Staphylococcus) and scalp dandruff. Posterior blepharitis affects the inner eyelid (the moist part that makes contact with the eye) and is caused by problems with the oil (meibomian) glands in this part of the eyelid. Two skin disorders can cause this form of blepharitis: acne rosacea, which leads to red and inflamed skin, and scalp dandruff (seborrheic dermatitis). Seborrheic blepharitis and meibomian gland dysfunction (meibomitis) are chronic types of blepharitis. Seborrheic blepharitis is more common in an older age group (mean age is around 50 years). Ulcerative blepharitis is an acute bacterial infection of the eyelid margin involving the lash follicles and the meibomian glands. Blepharitis often is associated with systemic diseases, such as rosacea and seborrheic dermatitis, as well as ocular diseases, such as dry eye syndromes, chalazion, trichiasis, conjunctivitis, and keratitis.

Symptoms of either forms of blepharitis include discomfort in the eye, burning and a sandy-gritty eye irritation, sensation of a foreign body in the eye. The eyelids appear red and swollen, with hard crusty material clinging to the base of the eyelashes and oily secretions along the edge of the eyelid. When removed crust may leave a bleeding surface. During sleep, the lids become glued together by dried oily secretions. Loss of eyelashes may also occur. Tear film problems is associated with blepharitis due to abnormal or decreased oil secretion, which may result in either excess tearing or dry eyes. There is often an overlap between blepharitis and keratoconjunctivitis sicca and chronic blepharitis may cause or exacerbate the condition. The overlapping symptoms reflect that conjunctivitis, blepharitis and keratoconjunctivitis sicca may co-exist and even develop from one another e.g. the clinical term blepharoconjunctivitis refers is the combination of conjunctivitis with blepharitis.

To date, the primary and prolonged treatment is thorough daily cleansing of the lid margins to remove the oily secretions that the bacteria feed on. Warm compresses with mild shampoo are often employed to loose the crusts, followed by gentle scrubbing of the eyelids. Ointments including antibiotics may also be used in controlling bacteria on the lids. Solutions for topical administration including corticosteroid and antibiotics may also be used in short-term treatment to control inflammation and symptoms of conjunctivitis. Such solutions are not recommended for long-term treatment. Blepharitis tends to reoccur and often develop into a chronic condition.

Taken together, the main treatment in the management of conjunctivitis, blepharitis and keratoconjunctivitis sicca overlap and are limited to warm or cold compresses, cleansing and use of artificial tears. In severe cases steroids and immunosuppressants are employed to control inflammation and antibiotics to control infection. The use of steroid medication in long-term treatment is not recommended due to potential adverse effects (e.g. thinning of skin). A medication possessing non-steroidal anti-inflammatory properties with no adverse effects is desirable for the long-term management of said diseases.

Flavonoids (or bioflavonoids) are a class of plant secondary metabolites which have received much attention for their potential medicinal properties associated with reduced risk of certain age related and human chronic diseases supported by epidemiological studies. There is experimental evidence associating flavonoids with anti-allergic, anti-inflammatory, anti-microbial and anti-cancer properties and demonstrating that flavonoids are strong antioxidants. Although several inflammation related enzymes including cyclooxygenase, lipoxygenase, PPAR, NOS, NKκB and NAG-1 have been proposed as molecular targets, the molecular mechanism still remains to be elucidated. Good sources of bioflavonoids include all citrus fruits, berries, onions, parsley, legumes, green tea, red wine, seabuckthorn, and cocoa.

Flavonoids are low molecular weight phenylbenzopyrones and belongs to the large group a group of vegetable chemical substances, the polyphenols, which are characterised by the presence of more than one phenol group per molecule. The group of flavonoids includes more than 5000 natural flavonoids that are categorised into five subgroups according to their chemical structure: flavonols, flavones, flavanones, flavan-3-ols and the anthocyanidins. The use of flavonoid compounds is known in the prior art.

### SUMMARY OF INVENTION

The object of the invention is the use of a composition comprising at least one flavonoid for the treatment of a disease or disorder of the eye and adnexa of the eye in an animal subject, including a human being. More particularly, this invention relates to a composition for the treatment of conjunctivitis, keratoconjunctivitis sicca, and blepharitis. The source of the flavonoids may be but are not restricted to citrus plants. The composition may be administrated as, but not restricted to, a topical formulation. The composition may be used in combination with an eyecleaner or eyewash, which may comprise at least one flavonoid. The invention furthermore relates to a pharmaceutical composition for the comprising at least one flavonoid for the treatment of a disease or disorder of the eye and adnexa of the eye in an animal subject, including a human being.

### DESCRIPTION OF DRAWINGS

FIG. 1: 27 year old male with severe blepharitis and partly eye-lid eczema, before treatment

FIG. 2: 27 year old male, after 4 weeks treatment twice a day with Bioflagel.

FIG. 3: 67 year old female with a long history of blepharitis.

FIG. 4: 67 year old female, after 6 weeks of treatment with Bioflagel.

FIG. 5: 83 year old male with severe blepharitis more or less all his life.

FIG. 6: 83 year old male, after 6 weeks of treatment with Bioflagel

FIG. 7: HPLC-DAD profile of bioflavoids extracted from Citrus aurantium. Wavelength 280 nm.

FIG. 8: HPLC-DAD profile of bioflavoids extracted from Citrus aurantium. Wavelength 360 nm.

### DETAILED DESCRIPTION OF THE INVENTION

### Conditions

According to the present invention the compositions are used for the treatment, amelioration or prevention of a medical condition in the eye and/or the adnexa of the eye including the diseases or disorders described below. In the present invention the eye and the adnexa of the eye comprises the area for application of the composition in question.

The treatment scheme may be prophylactic, thus the treatment may be administered in individuals at risk of acquiring the conditions described herein.

The composition according to the present invention is preferably used for the treatment or amelioration of blepharitis such as staphylococcal blepharitis, seborrhoeic blepharitis or allergic blepharitis. The composition may also be used for the treatment of blepharoconjunctivitis or neonatal conjunctivitis.

In one embodiment of the invention the composition is for the treatment of a disease or disorder selected from the group consisting of blepharitis, chronic blepharitis, Sjoegren's syndrome, ocular roseacea, conjunctivitis, keratoconjunctivitis sicca, blepharoconjunctivitis, and neonatal conjunctivitis.

In another preferred embodiment of the present invention, the composition is used in the treatment or amelioration of keratoconjunctivitis sicca, also known as dry eye syndrome (or simply dry eyes). The invention also relates to the treatment of keratoconjunctivitis sicca associated with an underlying systemic disease such selected form the group consisting of Sjögren's syndrome, systemic lupus erythematosus, rheumatoid arthritis, scleroderma, sarcoidosis, amyloidosis, hypothyroidism, and deficiency of vitamin A.

In another embodiment of the present invention, the composition is used in the treatment or amelioration of conjunctivitis. Conjunctivitis according to the present invention may be non-infectious or infectious.

Non-infectious conjunctivitis comprises allergic conjunctivitis (e.g. caused by pollen), chemical conjunctivitis (e.g. caused by splash injury of house hold or industrial chemicals), conjunctivitis caused by toxin(s) or burns and non-infectious conjunctivitis caused by underlying system diseases.

Accordingly, in one embodiment of the invention the composition is for the treatment of conjunctivitis caused by viral infection, bacterial infection, an allergen, an irritant, a chemical substance, or a toxin, or thermally induced.

Non-infectious conjunctivitis caused by underlying system diseases comprises Sjoegren's syndrome, Crohn's disease, ulcerative colitis or rheumatoid arthritis. Infectious conjunctivitis accounting for the majority of conjunctivitis cases comprise viral conjunctivitis (e.g. adenoviral infection) and bacterial conjunctivitis.

In one embodiment of the present invention concerns the treatment of ocular roseacea caused by underlying roseacea.

The conditions according to the present invention may be acute or chronic.

The subject for the treatment or amelioration of the disease comprises human beings and animals such as dogs, cats, horses, cows or sheep in the need thereof. In the preferred embodiment the subject is a human being.

A disease or disorder of the eye and/or the adnexa of the eye of a dog further include the conditions canine distemper and cherry eyes.

### Flavonoids

In one embodiment of the present invention the composition comprise at least one flavonoid selected from the group of polyphenols. In another embodiment the poly phenols includes a phenylbenzopyrone structure as known for the flavonoids. The polyphenol(s) may extracted from a any natural source such as a citrus bioflavonoid from citrus such as citrus aurantium or citrus bergamia, or a derivative of said isolated polyphenols, or the polyphenol may be synthetic.

In the preferred embodiment the composition comprise at least one bioflavonoid such as a citrus bioflavonoid. Said citrus bioflavonoid may be isolated from a citrus such as citrus bergamia or preferably citrus aurantium. Said citrus bioflavonoid(s) may be extracted from whole fruits (fresh or frozen). In another embodiment the citrus bioflavonoid(s) is extracted from fresh or frozen peel tissue of fruits, juice vesicle tissue of fruits, flavedo tissue of fruits, albedo tissue of fruits and segment epidermis tissue of fruits.

Preferably the bioflavonoids are extracted from whole citrus fruits such as fruits of citrus aurantium as for example described in example 12.

The size of the citrus fruits according to the invention is preferably no more than 60 mm, more preferably 40 to 55 mm. In one preferred embodiment of the invention the fruits are frozen before further processing in order to assist the release bioflavonoids from the fruits. Thawing of the fruits before the processing is preferably performed using air-ventilated containers keeping the temperature at the surface of the fruits below 5° C. According to the invention fruits are sliced into pieces with dimension in around 5×10×20 mm. In one embodiment of the invention the bioflavonoids extracted by at least four repeated rounds of extraction in water keeping the temperature below 20° C. The juice from the extraction is filtered (micro membrane ultrafiltration) using membranes such as PVDF (Polyvinylidene Fluoride), PSO (Polysulfon) membranes. In one preferred embodiment the UF membrane is a PVDF membrane. The cut-off value of the membrane (such as a PVDF membrane) may be in the range 3-5 kDa to 500 kDa, such as in the range 5 to 25 kDa, or such as in the range 20 to 200 kDa, for example 20 kDa membranes, 100 kDa membranes or 200 kDa membranes. In one preferred embodiment the membranes is a 100 kDa PVDF membrane. The filtration is performed at a temperature below 20° C., such as in the range of 6 to 20° C., preferably in the range of 6 to 14° C. In one preferred embodiment of the invention the temperature is in the range of 10-12° C. The bioflavonoids are separated on columns using absorbents suitable for the application such as Dowex and Amberlite absorbents. In one preferred embodiment of the invention the absorbent is Amberlite XAD7HP. The product of the column separation is an alcohol-water elution (such as an ethanol-water elution) containing the bioflavonoids from which the alcohol is evaporated using a vacuum evaporator. According to the invention the remaining solvent (mainly water) may be evaporated using a spay-drier to obtain a dry powder.

The bioflavonoids used for the preparation of the composition according to the invention may comprise at least one bioflavonoid, said bioflavonoid may be selected from the group consisting of flavonols, flavanones, flavones, flavan-3-ols, and anthocyanidins. According to the invention the composition may comprise at least one bioflavonoid independently selected from the group consisting of quercetin, neoeriocitrin, naringin, and neohesperidin. According to the invention the content of neoeriocitrin, naringin, neohesperidin may account for more than 40% of the total bioflavonoids in the composition such as more than 50%, for example more than 60%, such as more than 70%, for example more than 80%, such as more than 90% of the total bioflavonoids in the composition. In one preferred embodiment according to the invention neoeriocitrin, naringin, neohesperidin account (on per weight) for 85% of the total bioflavonoids in the composition (neoeriocitrin 9%, naringin 36%, neohesperidin 40%). The analysis of said extracted bioflavonoids is shown in example 13.

In one embodiment of the invention the at least one flavonoid is independently selected from the group consisting of the subgroup of flavonols, the subgroup of flavanones, the subgroup of flavones, the subgroup of flavan-3-ols, and the subgroup of anthocyanidins.

In another preferred embodiment, said composition comprises at least one second active ingredient. Said second active ingredient may be any second active ingredient.

### Formulation and Administration of Compositions and Pharmaceutical Compositions

The composition may be formulated in a number of different manners, depending on the purpose of the particular composition/pharmaceutical composition and the type of administration. It is well within the scope of a person skilled in the arts to formulate compositions that are in accord with the preferred type of administration.

One preferred embodiment of the present invention is to provide a composition formulated for topical application on a local, superficial and restricted area in the eye and the adnexa of the eye comprising at least one pharmaceutically acceptable additive and at least one flavonoid.

In said above-mentioned embodiment, it is preferred that the composition is formulated as an ointment, a lotion, a crème, a bath admixture, a gel, a paste, a milk, a suspension, an aerosol, a spray, a film, a foam, a serum, a swab, a pledget, a pad, a patch, a powder, a paste, a liniment, viscous emulsion, or another formulation which is appropriate for topical administration.

Such compositions for topical administration may further include physiologically acceptable components such as carriers, surfactants, preservatives, stabilizing agents, buffers, excipients and emulsifiers suited for this type of administration. Suitable components for topical delivery systems are preferably chosen from components that do not cause excessive or unavoidable irritation or pain to the recipient. Carriers include diluents and provide the medium in which the pharmaceutical constituents are dissolved, dispersed or distributed.

The composition according to the invention may comprise, but are not restricted, a carrier such as an aqueous liquid base, nonaqueous liquid base, water soluble gel, a mineral oil base, emulsion, ointment, crème, gel or lotion, suspension of solid particles in a liquid.

The topical availability of drugs depends on two contrasting factors: their ability to dissolve in the carrier (gel, creme—hydrophilic), and their ability to permeate the skin barrier (ie, the stratum corneum—hydrophobic), thus requiring a unique hydrophobic-hydrophilic balance. Formulations require addition of excipients, such as permeation enhancers and solubilizers to facilitate either or both of the transport processes (dissolution into vehicle and diffusion across skin). Additives, such as alcohols, fatty alcohols, fatty acids, mono- di- or tri-glycerides, glycerol monoethers, cyclodextrin and derivatives, polymers, bioadhesives, terpenes, chelating agents and surfactants have been disclosed to increase transdermal delivery of drugs. It is within the present invention to make use of such excipients.

Any method, not limited to the above-mentioned, for increasing transdermal delivery is within the scope of the present invention. The therapeutic composition according to the present invention may therefore comprise surfactants such as ionic and/or non-ionic surfactants. Suitable non-ionic surfactants include for example: fatty alcohol ethoxylates (alkylpolyethylene glycols); alkylphenol polyethylene glycols; alkyl mercaptan polyethylene glycols; fatty amine ethoxylates (alkylaminopolyethylene glycols); fatty acid ethoxylates (acylpolyethylene glycols); polypropylene glycol ethoxylates (Pluronic); fatty acid alkylolamides (fatty acid amide polyethylene glycols); alkyl polyglycosides, N-alkyl-, N-alkoxypolyhydroxy fatty acid amide, in particular N-methyl-fatty acid glucamide, Poloxamer 188, sucrose esters; sorbitol esters, esters of sorbitol polyglycol ethers and lecithin. Ionic surfactants include for example sodium lauryl sulfate, sodium laurate, polyoxyethylene-20-cetylether, Laureth-9, sodium dodecylsulfate (SDS) and dioctyl sodium sulfosuccinate.

Alcohols include, but are not limited to, ethanol, 2-propanol and polyols such as polyethylene glycol (PEG), propylene glycol, glycerol, propanediol.

Methods for enhancing drug delivery through topical administration may be applied with the present invention, and include any means of increasing absorption, minimizing metabolism, and/or prolonging the half-life of the active ingredient of the composition, such as flavonoid. Such means include the use of transporters of the type liposomes, ISCOMs, nano-particles, microspheres, hydrogels, organogels, polymers or other micro-encapsulation techniques.

Bioadhesives within the scope of the present invention for use in topical delivery include adhesives of the skin and mucous tissue such as mucin binding and/or epithelial tissue binding polymers.

In embodiments of the invention wherein the composition is formulated as a gel or gel-like substance, creme or viscous emulsions it is preferred that said composition comprises at least one gelling component, polymer or other suitable agent to enhance the viscosity of the composition. Any gelling component known to a person skilled in the art, which has no detrimental effect on the area being treated, and is applicable in the formulation of compositions and pharmaceutical compositions for topical administration to the skin, eye or mucous can be used. For example, the gelling component may be selected from the group of: acrylic acids, carbomer, carboxypolymethylene, such materials sold by B.F. Goodrich under the trademark Carbopol (e.g. Carbopol 940), polyethylene-polypropyleneglycols, such materials sold by BASF under the trademark Poloxamer (e.g. Poloxamer 188), a cellulose derivative, for example hydroxypropyl cellulose, hydroxyethyl cellulose, hydroxyethylene cellulose, methyl cellulose, carboxymethyl cellulose, alginic acid-propylene glycol ester, polyvinylpyrrolidone, veegum (magnesium aluminum silicate), Pemulen, Simulgel (such as Simulgel 600, Simulgel EG, and simulgel NS), Capigel, Colafax, plasdones and the like and mixtures thereof.

A gel or gel-like substance according to the present invention comprises for example less than 10% w/w water, for example less than 20% w/w water, for example at least 20% w/w water, such as at least 30% w/w water, for example at least 40% w/w water, such as at least 50% w/w water, for example at least 75% w/w water, such as at least 90% w/w water, for example at least 95% w/w water. Preferably said water is deionised water.

In one embodiment the composition is formulated as described in example 11 (Bioflagel UDV).

In one embodiment the composition is formulated as an ointment. Any ointment components known to a person skilled in the art, which has no detrimental effect on the area being treated, and is applicable in the formulation of compositions and pharmaceutical compositions for topical administration to the skin, eye or mucous can be used. For example, one carrier may be a petrolatum carrier.

In one embodiment the composition is formulated so it is a liquid comprising a least one flavonoid in solution or in suspension. The composition may be formulated in the any liquid form suitable for topical application such as eye-drops, artificial tears, eye washes, or contact lens adsorbents comprising a liquid carrier such as a cellulose ether (e.g. methylcellulose).

The liquid may be any useful liquid, however it is frequently preferred that the liquid is an aqueous liquid. It is furthermore preferred that the liquid is sterile. Sterility may be conferred by any conventional method, for example filtration, irradiation or heating.

The liquid may comprise one or more lipophile vehicles, for example one or more lipophile vehicle suitable for controlled release of flavonoids.

The composition and pharmaceutical compositions containing at least one flavonoid may be prepared by any conventional technique, e.g. as described in Remington: The Science and Practice of Pharmacy 1995, edited by E. W. Martin, Mack Publishing Company, 19th edition, Easton, Pa.

The composition according to the invention may be administered once or several times per day, such as e.g. two, three, four or five times per day. In a preferred embodiment of the invention the composition is administered twice a day. In another preferred embodiment of the invention the composition is administered once a day. In another embodiment of the invention the administration of a composition according to the invention is combined with the use of an eye cleaner, such as e.g. an eye cleaner comprising flavonoids/bioflavonoids as described herein for a composition according to the invention.

The treatment period may vary depending on the specific disease or condition treated. However, typically the treatment period is for at least 2 weeks, such as e.g., at least 3 weeks, at least 4 weeks or at least 5 weeks. In a preferred embodiment of the invention subjects in need of treatment are treated with a composition according to the invention for a period of about 3-6 weeks, this treatment may optionally be combined with the use of an eye cleaner as described above. In a more preferred embodiment of the invention the composition according to the invention is applied for a period of about 3 weeks, this treatment may optionally be combined with the use of an eye cleaner as described above.

For the treatment of blepharitis (seborrheic, rosacea, psoriasis or atopic dermatitis) the preferred treatment is an application of the composition according to the invention to the eyelids twice daily for a period of about 4-6 weeks. This treatment may optionally be combined with cleansing of the eyelid with an eye cleaner (eyelid-cleanser).

As mentioned previously herein, several diseases or conditions of the eye and/or the adnexa of the eye are chronic diseases that will recur repeatedly. For example, chronic blepharitis will typically recur 6-7 times a year. However, the composition according to the invention can remove the symptoms of blepharitis after a treatment period of about 3-6 weeks and at the same time prolong the period before recurrence, such that the symptoms typically recur 4 times a year.

Accordingly, in a preferred embodiment of the invention the use of a composition according to the invention can reduce recurrence of chronic diseases or condition of the eye and/or the adnexa of the eye, for example by at least 10%, at least 25%, or at least 40%.

The pharmaceutical acceptable additives may be any conventionally used pharmaceutical acceptable additive, which should be selected according to the specific formulation, intended administration route etc. For example the pharmaceutical acceptable additives may be any of the additives mentioned in Nema et al, 1997. Furthermore, the pharmaceutical acceptable additive may be any accepted additive from FDA's “inactive ingredients list”, which for example is available on the internet address http://www.fda.gov/cder/drug/iig/default.htm.

Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate salts. Suitable base salts include, but are not limited to, aluminum, calcium, lithium, magnesium, potassium, sodium, zinc, and diethanolamine salts.

It is contained within the present invention that at least one pharmaceutically acceptable additive is a buffer. For some purposes it is often desirable that the composition comprises a buffer, which is capable of buffering a solution to a pH in the range of 5 to 9, for example pH 5 to 6, pH 6 to 8 or pH 7 to 7.5.

However, in other embodiments of the invention the pharmaceutical composition may comprise no buffer at all or only micromolar amounts of buffer.

The buffer may for example be selected from the group consisting of TRIS, acetate, glutamate, lactate, maleate, tartrate, phosphate, citrate, carbonate, glycinate, histidine, glycine, succinate and triethanolamine buffer. Hence, the buffer may be K₂HPO₄, Na₂HPO₄ or sodium citrate.

In a preferred embodiment the buffer is a TRIS buffer. TRIS buffer is known under various other names for example tromethamine including tromethamine USP, THAM, Trizma, Trisamine, Tris amino and trometamol. The designation TRIS covers all the aforementioned designations.

The buffer may furthermore for example be selected from USP compatible buffers for parenteral use, in particular, when the pharmaceutical formulation is for parenteral use. For example the buffer may be selected from the group consisting of monobasic acids such as acetic, benzoic, gluconic, glyceric and lactic, dibasic acids such as aconitic, adipic, ascorbic, carbonic, glutamic, malic, succinic and tartaric, polybasic acids such as citric and phosphoric and bases such as ammonia, diethanolamine, glycine, triethanolamine, and TRIS.

In some embodiments of the invention the pharmaceutically acceptable additives comprise a stabiliser. The stabiliser may for example be a detergent, an amino acid, a fatty acid, a polymer, a polyhydric alcohol, a metal ion, a reducing agent, a chelating agent or an antioxidant, however any other suitable stabiliser may also be used with the present invention.

For example the stabiliser may be selected from the group consisting of poloxamers, Tween-20, Tween-40, Tween-60, Tween-80, Brij, metal ions, amino acids, polyethylene glucol, Triton, and ascorbic acid.

Furthermore, the stabiliser may be selected from the group consisting of amino acids such as glycine, alanine, arginine, leucine, glutamic acid and aspartic acid, surfactants such as polysorbate 20, polysorbate 80 and poloxamer 407, fatty acids such as phosphotidyl choline ethanolamine and acethyltryptophanate, polymers such as polyethylene glycol and polyvinylpyrrolidone, polyhydric alcohol such as sorbitol, mannitol, glycerin, sucrose, glucose, propylene glycol, ethylene glycol, lactose and trehalose, antioxidants such as ascorbic acid, cysteine HCL, thioglycerol, thioglycolic acid, thiosorbitol and glutathione, reducing agents such as several thiols, chelating agents such as EDTA salts, glutamic acid and aspartic acid.

The pharmaceutically acceptable additives may comprise one or more selected from the group consisting of isotonic salts, hypertonic salts, hypotonic salts, buffers and stabilisers.

In preferred embodiments other pharmaceutically excipients such as preservatives are present. In one embodiment said preservative is a parabene, such as but not limited to methyl parahydroxybenzoate or propyl parahydroxybenzoate.

Compositions and pharmaceutical compositions according to the present invention, comprise at least one flavonoid as an active ingredient. The concentration of flavonoid in said compositions may vary according to the type of administration they are formulated for. The compositions may comprise 0.1 ng/ml to 10 mg/ml, preferably 10 ng/ml to 1 mg/ml, such as 100 ng/ml to 100 μg/ml, preferably 100 ng to 10 μg/ml flavonoid. The compositions may comprise 0.1 ng/ml to 1.0 ng/ml, for example 1.0 ng/ml to 10 ng/ml, for example 10 ng/ml to 100 ng/ml, for example 100 ng/ml to 1.0 μg/ml, for example 1 μg/ml to 10 μg/ml, for example 10 μg/ml to 100 μg/ml, for example 100 μg/ml to 1.0 mg/ml, for example 1 mg/ml to 10 mg/ml, for example 10 mg/ml to 100 mg/ml flavonoid.

Compositions and pharmaceutical compositions for topical delivery, according to the present invention, comprise at least one flavonoid as an active ingredient. The compositions may comprise 0.01 to 10 wt % of flavonoid, preferably 1 to 5 wt %, more preferably 1 to 4 wt %, or most preferably 0.1 to 2% by weight of the flavonoids/bioflavonoids.

According to the present invention “a pharmaceutical effective dosage” of the composition refers to the amount necessary to induce the desired biological effect on the subject in need of treatment.

The compositions and pharmaceutical compositions according to the present invention may be administrated once or more than once a day, for example they may be administered in the range of 2 to 10 times a day, such as 2 to 7 times, for example 2 to 5 times, such as 2 to 4 times, such as 2 to 3 times a day.

The compositions according to the present invention may be administrated to the subject for a period of treatment of one or more than one week such as two weeks, three weeks, four weeks, five weeks, six weeks, seven weeks, eight weeks or more than eight weeks. The treatment may be repeated on subjects, who relapse.

The compositions according to the present invention (such as Bioflagel UDV, Example 10) may be administered in combination with an eyecleaner such as the Bioflagel eyecleaner UDV (Example 11), which is based upon a natural cleaning liquid plus the addition of the above described Citrus Aurantium bioflavonoids.

In one embodiment of the invention, the subject in need is treated with an eyecleaner or eyewash prior treatment with the composition according to the invention.

A further aspect of the present invention relates to a pharmaceutical composition as defined above for a composition.

A further aspect of the present invention relates to a method of treating or ameliorating a disease or disorder of the eye and/or adnexa comprising administration to an animal subject including a human being in need thereof an effective dosage of a composition or a pharmaceutical composition as defined herein above.

It is within the scope of the present invention to supply compositions, and uses thereof, comprising flavonoids for the treatment of clinical conditions described above involving an infection or an increased risk of acquiring an infection. For example, but not limited to, clinical conditions involving infection, or is at risk of being infection, by a microbial species. In one embodiment of the invention flavonoids is co-administered with at least one second active ingredient. Preferably flavonoids and said least one second active ingredient are present in the same composition, or they may be supplied in a kit of parts. Preferably, said second active ingredient is a disinfectant (e.g. pharmaceutically acceptable salt of boric acid such as sodium borate), antimicrobial substance, for example an antibiotic, antifungal, antiparasitic or antiviral agent.

### EXAMPLES

### Example 1

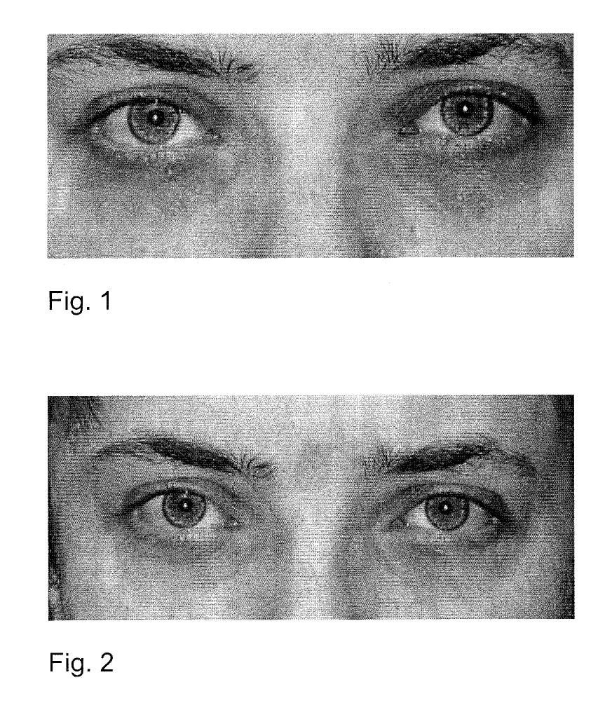

27 year old male with severe blepharitis and partly eye-lid eczema (FIG. 1). No significant effect of blephagel and steroid. 4 weeks treatment with Bioflagel twice a day had significant effect with no further symptoms (FIG. 2).

### Example 2

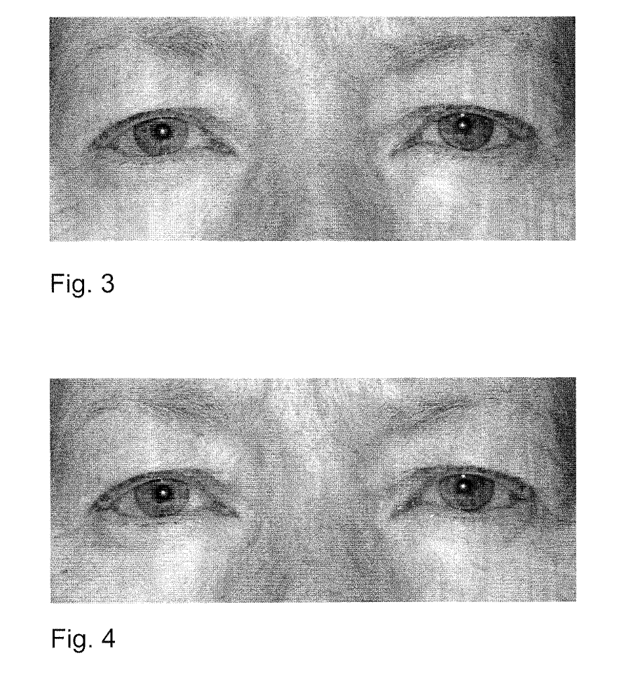

67 year old female with a long history of blepharitis—tried different products with no significant effect (FIG. 3). She had 6 weeks of treatment with Bioflagel and eye-cleaner—all symptoms disappeared (FIG. 4)—no re-treatment so far.

### Example 3

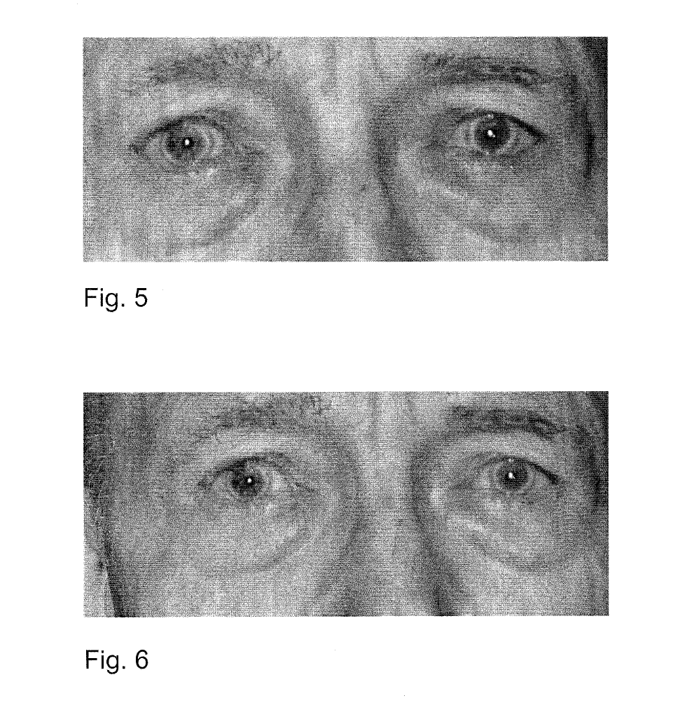

83 year old male with severe blepharitis more or less all his life (FIG. 5). Free of symptoms after Bioflagel twice a day in a 6 week period (FIG. 6)—so far no re-treatment.

### Example 4

63 year old female with long-time severe blepharitis. Responds with rice of the intraocular pressure after treatment with steroids. Free of symptoms after 6 weeks treatment with Bioflagel combined with the eye-cleaner twice a day—no recurrency.

### Example 5

88 year old female with chronical blepharitis—4 weeks treatment with Bioflagel and eye-cleaner made her free of symptoms. No re-treatment so far.

### Example 6

73 year old male with blepharitis/conjunctivitis. 5 weeks treatment with Bioflagel and eye-cleaner cleared his symptoms.

### Example 7

58 year old male with blepharitis and eye-lid eczema as well. Had 5 weeks treatment with Bioflagel and eye-cleaner. All symptoms disappeared with no recurrency so far.

### Example 8

76 year old female with long-time severe blepharitis. Free of symptoms after treatment with Bioflagel and eye-cleaner twice a day for a 4 week period.

### Example 9

84 year old female with chronical blepharitis. 5 weeks treatment with Bioflagel made her symptoms disappear. No re-treatment so far.

### Example 10

### Bioflagel UDV

| Ingrediens | CAS no. | % | Active |
|---|---|---|---|
| Aqua | 7732-18-5 | 87.4600 | 87.4600 |
| Poloxamer 188 | 9006-11-6 | 5.0000 | 5.0000 |
| Triethanolamine | 102-71-6 | 2.6000 | 2.3400 |
| PEG-75 | 25322-68-2 | 2.0000 | 2.0000 |
| Sodium Borate | 1330-43-4 1303-96-4 | 0.8400 | 0.8400 |
| Carbomer | 9003-01-4 | 0.8000 | 0.8000 |
| Methylparaben | 99-75-3 | 0.3000 | 0.3000 |
| Bioflavonoids | | 1.0000 | 0.1600 |

### Example 11

### Bioflagel Eyecleaner UDV

| INCI | CAS no. | % | Active |
|---|---|---|---|
| Aqua (85° C.) | 7732-18-5 | 39.7700 | 39.7700 |
| Sodium Laureth Sulfate | 3088-31-1 | 44.4400 | 11.9988 |
| Glycerin | 56-81-5 | 3.5000 | 3.5000 |
| Cocamidopropyl Betaine | 61789-40-0 | 6.3800 | 2.5520 |
| Sucrose Cocoate | 91031-88-8 | 2.3000 | 1.4950 |
| TEA-Lauryl Sulfate | 90583-18-9 | 2.5000 | 1.0500 |
| Phenoxyethanol | 122-99-6 | 0.4000 | 0.4000 |
| Sodium Chloride | 7647-14-5 | 0.4000 | 0.4000 |
| Citric Acid | 5949-29- | 0.2000 | 0.2000 |
| Sodium Benzoate | 532-32-1 | 0.1000 | 0.1000 |
| Bioflavonoids | | 0.0100 | 0.0016 |

### Example 12

### Processing of Citrus Fruits to Bioflavonoids

### Harvesting of Citrus Fruits.

The fruits are preferably harvested when they are still green and contain higher levels of flavonoids than the yellow later state fruits. Fruits with a diameter preferably less than 60 mm are used and most preferred fruits in the range of 40 to 55 mm.

### Freezing of the Fruits.

The fruits are then frozen to −20° C., or lower in order to disrupt the cell walls allowing release of flavonoids by diffusion. The fruits remain frozen until further processing.

### Thawing of Fruits.

Batches of frozen fruits are transferred to air-ventilated containers for thawing. The flow of air around the fruits transfers heat, so that after some time the entire fruit is thaw. It is preferred that temperature is below 5° C. at the surface; but melted at the core of the fruits, when the fruits are transferred for slicing.

### Slicing of Fruits.

The fruits are sliced in a commercial slicer making pieces of 5×10×20 mm. It has been found that this size gives a reasonable time for the extraction. Pieces of larger dimension increase the time of extraction. Fruits processed into smaller dimension there will also include finely divided matter which has a tendency to clog the filters.

### Extraction of Flavonoids.

The extraction is done according to a principle known from the beet sugar industry, before the invention of the continuos devices. A number of tanks equipped with an appropriate stirrer and filter means are filled with the sliced fruit and water, and the slurry is circulated by means of a stirrer. The stirring is done in order to increase Reynold number so much that the flow is turbulent, and thus increase mass transfer. The juice from said process is transferred to another tank filled with sliced fruit for another round of extraction. Preferably the number of extraction steps is more than four. The process of repeating extractions allows the extraction of almost all flavonoids into the juice and obtaining a concentration of flavonoids optimal for further processing. During the extraction the temperature is kept below 20° C. in order to minimize the contamination of the juice with other substances causing problems in the further processing.

### Membrane Micro/Ultra Filtration.

Membrane Micro/ultra filtration step is performed in order to protect the following separation in an adsorbent column. Membranes allowing the passage of flavonoids and retaining larger molecular matters and particles are preferred.

Cut-off values in the range of 20-200 kDa has proven to work well, even smaller pores may work well; but there are limitations in the commercial availability of membranes suitable for this particular type of juice. The filtration is performed a temperatures preferably in the range of 6 to 14° C.

### Adsorption of Flavonoids to an Adsorbent.

Absorbents selectively retaining the flavonoids in interest are chosen for the specific application. Available commercial adsorbents include Dowex and Amberlite such as Amberlite XAD7HP used in this application. The column is a packed column without stirring.

Once the absorbent material is saturated with flavonoids, the column is washed with water in order to wash out material attached to the surface of the adsorbent material. Drained from water the flavonoids are eluted from the column using ethanol into an ethanol/water solution contain 0.7% to 1% flavonoids, which is used for further processing.

Evaporation of Ethanol/Water from the Solution of Flavonoids.

The solution of flavonoids in ethanol/water is further concentrated in a vacuum evaporator operated at a temperature of around 45° C., until it reaches a Bx of 25-30. During the evaporation the majority of alcohol is evaporated. The final flavonoid water solution contains only minor traces of ethanol.

### Spray Drying of Flavonoid Solution.

The concentrated juice transferred to a spray-drier for evaporation of the solvent. Nozzles at the top spray-drier generates small droplets, and hot air (180° C.) is blown into the device in counter stream causing evaporation of the solvent (mainly water). The temperature at the core of the droplet is kept low due the heat consumption of the evaporation. The exhaust air is around 90° C. Flavonoid powder is collected from the spray-drier at the end of the process.

### Example 13

### The Active Component of Bioflagel: Citrus Aurantium Bioflavonoids

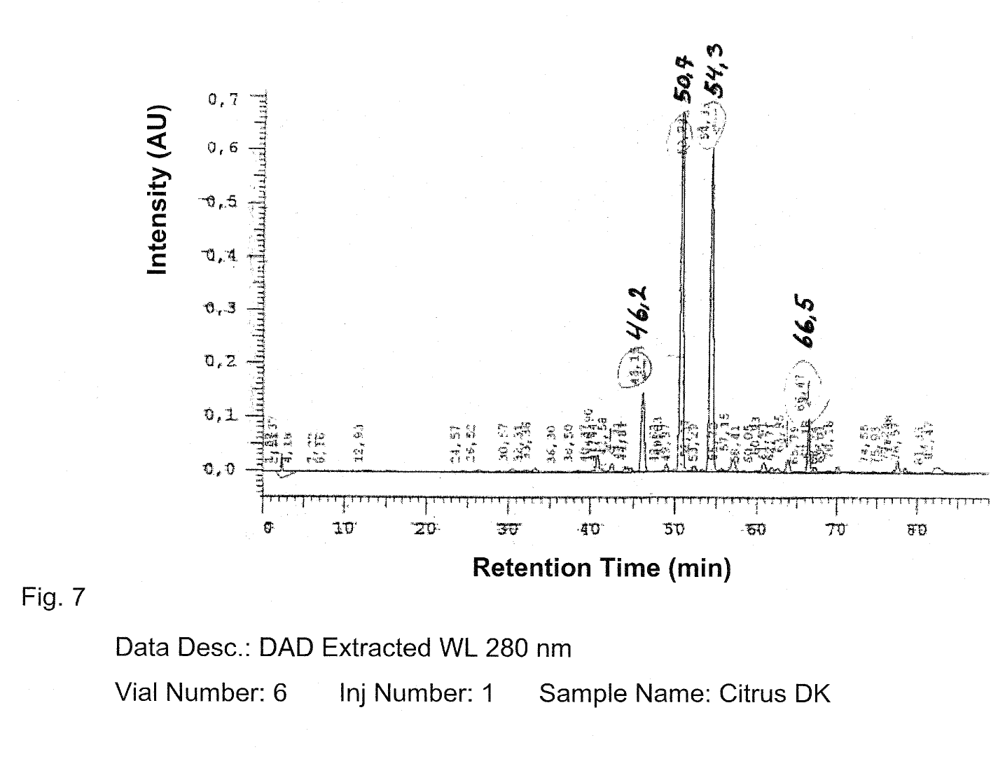

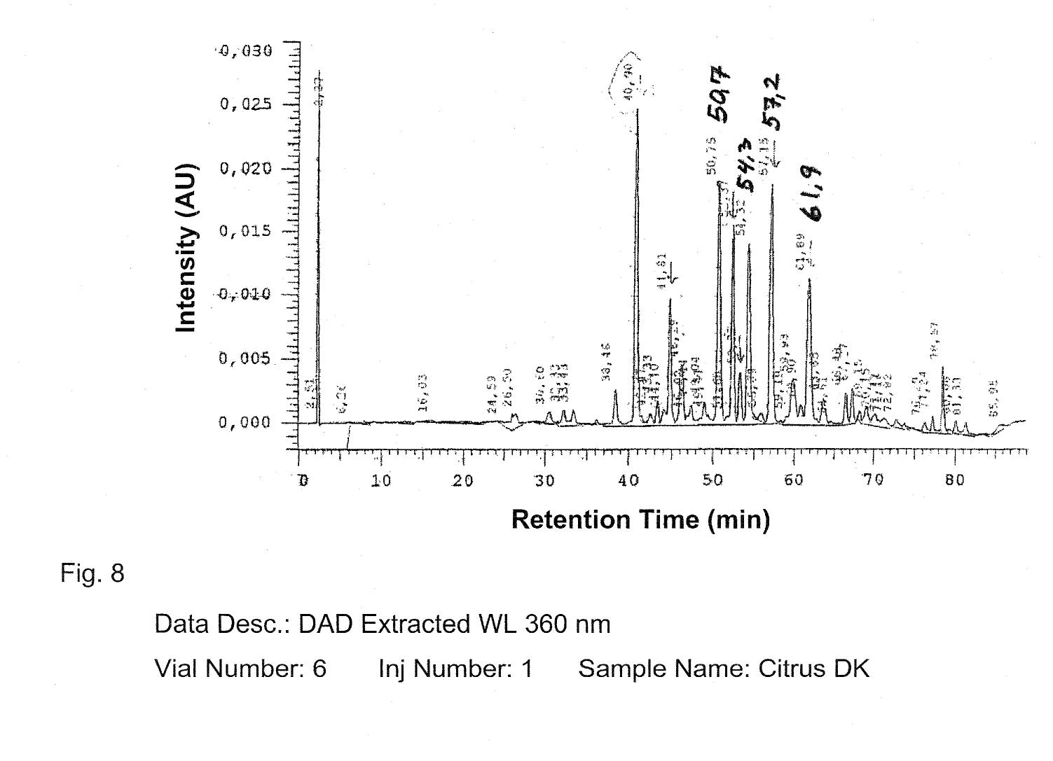

Samples of Citrus Aurantium (Spanish origin) were analyzed by HPLC-DAD (High Performance Liquid Chromatography-Diode Array Detection). In general flavonoides absorb UV-visible radiation at approximately 280 nm (flavanoles or flavanons) and/or 360 nm (flavons). The HPLC-chromatograms from the analysis and the purification procedure of Citrus flavonoides are therefore presented at these two wavelengths. As can be seen from the enclosed HPLC-chromatograms 6 different distinctive flavonoids in high quantities and several other flavonoids in very small quantities were demonstrated. At 280 nm four main peaks are observed: 46.2, 50.7, 54.3 and 66.5 minutes (FIG. 7), whereas at 360 nm four main peaks are observed: 50.7, 54.3, 57.2 and 61.9 minutes (FIG. 8). The 50.7, 54.3 peaks are observed at both wavelengths. The data from the HPLC analysis show that the Citrus Aurantium sample mainly include four flavanols/flavanons and two flavons, where the flavones are present in remarkable lower concentration than the flavanoles/flavanones.

Flavonoids corresponding to five out of the six main peaks from the HPLC-DAD analysis were isolated and analyzed by NMR. Three of the five isolated flavonoids were identified as neoeriocitin, narigin and neohesperidin accounting for 9.388%, 36.212% and 39.891% of the total flavonoids of the sample (on per weight).

### Sample Preparation and HPLC-DAD Analysis:

29.4 mg citrus sample was dissolved in 10 ml methanol (SAMPLE). SAMPLE was diluted by a factor 4 (400 μl SAMPLE to 1.6 ml methanol) and analysed by HPLC-DAD. Equipment: Shimadzu HPLC, Merck Diode Array Detektor (DAD), LiChrospher 100 RP-18 Column (No. 924112). Oven temp. 35° C.; Injection volume: 20 μl; flow: 1 ml/min; fixed wavelength: either 280 or 360 nm; Scan wavelength: 230-500 nm.

Eluent A: 5% formic acid in water.

Eluent B: 50% methanol, 15% acetic acid, in water.

### HPLC-Gradient:

| Minuttes | 0.01 | 5 | 20 | 35 | 45 | 55 | 75 | 80 | 90 | 90.1 |
|---|---|---|---|---|---|---|---|---|---|---|
| Conc. eluent | 0 | 3 | 10 | 25 | 40 | 40 | 100 | 0 | 0 | stop |
| B, % | | | | | | | | | | |

### Example 14

### Ongoing Clinical Trial: Investigation of the Symptom Reductive Effect of Bioflagel Compared to Blephagel

### Aim:

The purpose of the study is to investigate the symptom reductive effect of Bioflagel, eye gel, compared to Blephagel, eye gel, in patients suffering from blepharitis.

### Design:

Single center open clinical trial. 50 patients with symptoms of blepharitis and which have given their written consent are included. The patients are divided in two treatment groups, i.e. one treated with Bioflagel, eyecare gel, and one with Blephagel, gel.

### Experimental Protocol:

The experimental population is recruited from medical specialist practice. Interested patients receive verbal and written information about the study.

### Visit 1:

The written information is distributed, and after written and verbal consent the patient is included in the study.

The intraocular pressure is measured, a visual test is performed, and a picture is taken.

Patient form is filled in with subjective description of the symptoms.

A diary is handed out for registration of symptoms in a 4 week period.

The patients are randomized for either Bioflagel or Blephagel treatment.

Treatment with the appropriate eye gel twice daily begins.

Visit 2—after 4 Weeks:

The patient is questioned about any adverse effects.

The diary is inspected.

If symptoms still are present, a diary for additional 8 weeks is handed out, and treatment with the appropriate eye gel continues.

If the patient is free of symptoms a picture is taken of the eyes, the intraocular pressure is measured, and a visual test is performed. The physician fill in the patient form and the patient's participation is finalised.

Visit 3—after 12 Weeks:

The patient is questioned about any adverse effects.

The diary is inspected.

A picture is taken of the eyes, the intraocular pressure is measured, and a visual test is performed.

The physician fill in the patient form and the patient's participation is finalised.

### Bioflagel Eyecare Gel

| Ingrediens | CAS no. | % |
|---|---|---|
| Aqua | 7732-18-5 | 87.4600 |
| Poloxamer 188 | 9006-11-6 | 5.0000 |
| Triethanolamine | 102-71-6 | 2.6000 |
| PEG-75 | 25322-68-2 | 2.0000 |
| Sodium Borate | 1330-43-4 1303-96-4 | 0.6400 |
| Sodium hyaluronate | 9067-32-7 | 0.2000 |
| Carbomer | 9003-01-4 | 0.8000 |
| Methylparaben | 99-75-3 | 0.3000 |
| Bioflavonoids | 61788-55-4 | 1.0000 |

### Blephagel, Gel

(commercial available composition used for comparison, not according to the invention) Ingredients: Water, poloxamer 188, PEG-75, sodium borate, carbomer, methylparaben

### Results so Far:

The majority of the 50 patients have been included in the study. So far the results from the patients that have concluded the study are as follows:

All patients treated with Bioflagel have experienced a significant improvement of their condition, whereas only ¼ of the patients in the treatment group with Blephagel have experienced a change.

1.-57. (canceled)

58. A composition for the treatment or amelioration of a disease or disorder of the eye and/or the adnexa of the eye in an animal subject, including a human being, the composition comprising the bioflavonoids neoeriocitrin, naringin, and neohesperidin, wherein these bioflavonoids account for more than 70% of the total bioflavonoids in the composition.

59. The composition according to claim 58, wherein the disease is blepharitis, staphylococcal blepharitis, seborrhoeic blepharitis or allergic blepharitis.

60. The composition according to claim 58, wherein the disease or disorder is selected from the group consisting of blepharitis, chronic blepharitis, Sjoegren's syndrome, ocular rosacea, conjunctivitis, keratoconjunctivitis sicca, blepharoconjunctivitis, and neonatal conjunctivitis.

61. The composition according to claim 58, wherein the disease is conjunctivitis and is caused by viral infection, bacterial infection, an allergen, an irritant, a chemical substance, or a toxin, or is thermally induced.

62. The composition according to claim 58, wherein the disease is keratoconjunctivitis sicca and is associated with an underlying system disease selected from the group consisting of Sjoegren's syndrome, systemic lupus erythematosus, rheumatoid arthritis, scleroderma, sarcoidosis, amyloidosis, hypothyroidism, and deficiency of vitamin A.

63. The composition according to claim 58, wherein said subject is selected from the group consisting of human being, dog, cat, horse, cow and sheep.

64. The composition according to claim 63, wherein said subject is a human being.

65. The composition according to claim 63, wherein said subject is a dog.

66. The composition according to claim 65, wherein the disease is selected from the group consisting of keratoconjunctivitis sicca, canine distemper and cherry eyes.

67. The composition according to claim 58, wherein at least one of said bioflavonoids belongs to the group of polyphenols.

68. The composition according to claim 67, wherein at least one of said bioflavonoids is a synthetic polyphenol.

69. The composition according to claim 58, wherein at least one of said bioflavonoids belongs to the group of citrus bioflavonoids.

70. The composition according to claim 69, wherein said citrus bioflavonoid is extracted from citrus aurantium or citrus bergamia.

71. The composition according to claim 70, wherein said citrus bioflavonoid is extracted from the whole fruits of said citrus.

72. The composition according to claim 71, wherein the diameter of said fruits are preferably less than 60 mm, more preferably in the range of 40 to 55 mm.

73. The composition according to claim 72, wherein the bioflavonoids of said fruits are extracted in water.

74. The composition according to claim 73, wherein the extraction is performed by repeated rounds of extraction, obtaining a juice comprising said bioflavonoids.

75. The composition according to claim 74, wherein the temperature during the extraction is kept below 20° C.

76. The composition according to claim 75, wherein the juice is subjected to membrane micro/ultra filtration.

77. The composition according to claim 76, wherein the product obtained from said filtration is separated on a column comprising an absorbent.

78. The composition according to claim 70, wherein said citrus bioflavonoid is extracted from at least one tissue selected from the group consisting of peel tissue of fruits, juice vesicle tissue of fruits, flavedo tissue of fruits, albedo tissue of fruits and segment epidermis tissue of fruits.

79. The composition according to claim 69, wherein at least one flavonoid is a derivative of a citrus bioflavonoid.

80. The composition according to claim 58, wherein at least one flavonoid includes a phenylbenzopyrone structure.

81. The composition according to claim 58, wherein at least one flavonoid is independently selected from the group consisting of the subgroup of flavonols, the subgroup of flavanones, the subgroup of flavones, the subgroup of flavan-3-ols, and the subgroup of anthocyanidins.

82. The composition according to claim 58, wherein at least one flavonoid is independently selected from the group consisting of quercetin, neoeriocitrin, naringin, and neohesperidin.

83. The composition according to claim 58 comprising a disinfectant.

84. The composition according to claim 83, wherein said disinfectant is a pharmaceutically acceptable salt of boric acid.

85. The composition according to claim 84, wherein said salt of boric acid is sodium borate.

86. The composition according to claim 58 further comprising at least one pharmaceutically acceptable additive selected from the group consisting of isotonic salts, hypertonic salts, hypotonic salts, buffers and stabilisers.

87. The composition according to claim 58 further comprising a carrier selected from the group consisting of an aqueous liquid base, a nonaqueous liquid base, a water soluble gel, a mineral oil base, and a suspension of solid particles in a liquid.

88. The composition according to claim 58 which is a topical formulation.

89. The composition according to claim 58 which is for cutaneous application.

90. The composition according to claim 88, wherein the topical formulation is selected from the group consisting of cream, ointment, gel, lotion, liniment, viscous emulsion, powder, paste, film, foam, milk, suspension, aerosol, spray, serum, swab, pledget, pad, patch, and bath admixture.

91. The composition according to claim 90 which is a gel.

92. The composition according to claim 91 comprising less than 10% w/w water.

93. The comprising according to claim 91, wherein said gel comprises at least one gelling component.

94. The composition according to claim 93, wherein the gelling component is a carbomer.

95. The composition according to claim 94, wherein said carbomer is carbopol 940.

96. The composition according to claim 91, wherein said gel comprises at least one surfactant.

97. The composition according to claim 96, wherein said surfactant is Poloxamer 188.

98. The composition according to claim 91, wherein said gel comprises at least one preservative.

99. The composition according to claim 98, wherein said preservative is a parabene.

100. The composition according to claim 90, which further comprises an ointment base suitable for direct application to the eye.

101. The composition according to claim 100, wherein said ointment comprises a petrolatum-based carrier.

102. The composition according to claim 87, wherein the composition is a liquid formulation in the form of eye-drops, artificial tears, eye washes, or contact lens adsorbents.

103. The composition according to claim 102, wherein the composition comprises a liquid carrier suitable for application to the eye.

104. The composition according to claim 103, wherein said carrier comprises a cellulose ether.

105. The composition according to claim 104, wherein said cellulose ether is methylcellulose.

106. The composition according to claim 58, wherein the pH is in the range of pH 5 to 9.

107. The composition according to claim 58, wherein the composition comprises 0.1-2% by weight of the flavonoids.

108. The composition according to claim 58 which is for use in combination with an eye-cleaner or eye-wash.

109. The composition according to claim 108, suitable for use following administration of an eye-cleaner or eye-wash.

110. The composition according to claim 108, wherein said eye-cleaner or eye-wash comprises at least one of the bioflavonoids neoeriocitrin, naringin, and neohesperidin.

111. The composition according to claim 58, suitable for administration once a day or several times per day.

112. The composition according to claim 58 comprising or suitable for use in combination with a second active agent.

113. The composition according to claim 58, wherein the disease or disorder is blepharitis selected from the group consisting of seborrheic blepharitis, rosacea, psoriasis and atopic dermatitis.

114. Method of treating or ameliorating a disease or disorder of the eye and/or adnexa, the method comprising administration to an animal subject including a human being in need thereof an effective dosage of the composition as defined in claim 58.

115. A pharmaceutical composition comprising the composition as defined in claim 58.

116. A composition comprising the bioflavonoids neoeriocitrin, naringin, and neohesperidin, wherein these bioflavonoids account for more than 70% of the total bioflavonoids in the composition, and wherein the composition is a liquid formulation in the form of an eye wash.

117. A composition comprising as an active ingredient the bioflavonoids neoeriocitrin, naringin, and neohesperidin, wherein these bioflavonoids account for more than 70% of the total bioflavonoids in the composition.

118. A pharmaceutical composition comprising as an active ingredient the bioflavonoids neoeriocitrin, naringin, and neohesperidin, wherein these bioflavonoids account for more than 70% of the total bioflavonoids in the composition.

119. A composition for the treatment of rosacea, the composition comprising the bioflavonoids neoeriocitrin, naringin, and neohesperidin, wherein these bioflavonoids account for more than 70% of the total bioflavonoids in the composition.

120. A composition for the treatment of seborrheic dermatitis, the composition comprising the bioflavonoids neoeriocitrin, naringin, and neohesperidin, wherein these bioflavonoids account for more than 70% of the total bioflavonoids in the composition.

121. A composition for the treatment of psoriasis, the composition comprising the bioflavonoids neoeriocitrin, naringin, and neohesperidin, wherein these bioflavonoids account for more than 70% of the total bioflavonoids in the composition.

122. A composition for the treatment of atopic dermatitis, the composition comprising the bioflavonoids neoeriocitrin, naringin, and neohesperidin, wherein these bioflavonoids account for more than 70% of the total bioflavonoids in the composition.

123. The composition according to claim 94, wherein the gelling component is hyaluronic acid.
